(19) United States
(12) Patent Application Publication
(10) Pub. No.: US 2021/0295731 A1
(43) Pub. Date: Sep. 23, 2021

(54) INFORMATION PROCESSING APPARATUS, INFORMATION PROCESSING SYSTEM, INFORMATION PROCESSING METHOD, AND COMPUTER PROGRAM

(71) Applicant: National University Corporation Tokai National Higher Education and Research System, Nagoya-shi, Aichi (JP)

(72) Inventors: Hirofumi AOKI, Nagoya-shi, Aichi-ken (JP); Makoto INAGAMI, Nagoya-shi, Aichi-ken (JP); Aiko IWASE, Nagoya-shi, Aichi-ken (JP)

(21) Appl. No.: 17/266,463

(22) PCT Filed: Aug. 5, 2019
(86) PCT No.: PCT/JP2019/030696

§ 371 Date: Feb. 5, 2021

(30) Foreign Application Priority Data
Aug. 7, 2018 (JP) 2018-148260

(51) Int. Cl. G09B 9/052; G06K 9/00; G06T 7/70; G09B 19/00; G06F 3/01

(52) U.S. Cl. CPC G09B 9/052; G06K 9/00845; G06T 7/70; G06T 2207/30268; G06F 3/012; G06F 3/013; G06T 2207/30196; G09B 19/00

(57) ABSTRACT

An information processing apparatus including: a head information acquiring unit, a display controlling unit, a viewpoint information acquiring unit, and a risk assessing unit. The head information acquiring unit acquires head information specifying movement of the head of the examinee. The display controlling unit displays a simulated moving image including: a scene and a target object. The viewpoint information acquiring unit acquires viewpoint information for specifying the position of the viewpoint of the examinee on the simulated moving image while the image is displayed. The risk assessing unit assesses the risk based on the degree of coincidence between the position of the viewpoint of the examinee specified by the viewpoint information and the position of the target object at the time when the simulated moving image is displayed, and outputs assessment information indicating the result of the risk assessment.

### CROSS REFERENCE TO RELATED APPLICATION

This application is a U.S. national stage application of International Application No. PCT/JP2019/030696, filed on Aug. 5, 2019, which claims priority of Japanese patent application no. 2018-148260, filed on Aug. 7, 2018, the contents of which are incorporated herein by reference.

### TECHNICAL FIELD

The technology disclosed herein relates to an information processing apparatus for assessing a risk of moving of an examinee.

### BACKGROUND

It is useful to appropriately assess risks of drivers when driving an automobile and moving on a road. For example, when a driver's license is to be issued or renewed, such a driving risk assessment is performed to judge whether or not the driver's license can be issued or renewed on the basis of the result of the risk assessment, and appropriate education and training are conducted on the basis of the result of the risk assessment, resulting in reduction of the average risk of drivers and prevention of traffic accidents. In particular, elderly people and people having developed eye diseases such as glaucoma tend to have higher risks of driving because they may have narrowed or defective visual fields, while it is also possible to compensate for narrowed or defective visual fields by appropriately moving their head and eyes, and it is very useful to assess the driving risk in consideration of such compensation action.

Conventionally, as a method for assessing a driving risk, there has been known a method which includes: displaying a moving image simulating a visual field of a driver in front of an examinee by using an image display device (for example, liquid crystal display or projector); conducting a test in which an examinee answers, orally or by button operation, a hazard (automobiles, bicycles, and pedestrians, among others) included in the moving image; and assessing the driving risk of the examinee on the basis of the result of the test (for example, see non-patent document 1).

### NON PATENT REFERENCE

[Non-Patent Document 1] Masahiro TADA, five others, “An Analysis of Elderly Drivers' Hazard Perception on Expressway”, JSTE Journal of Traffic Engineering, February 2016, Vol. 2, No. 2, pp.A 75-A84.

The above-described conventional technology may not be possible to simulate natural driving conditions since the moving image is displayed in front of the examinee during the test. For example, in a scene of an intersection with poor visibility, the movement of the head and the eyes is important for checking right and left, but when the moving image is displayed in front of the examinee, the head and the eyes rotates oppositely, which is different from the movement of the head and the eyes in an actual driving environment. In addition, according to the above-described conventional technology, the examinee answers orally or by button operation that he/she has recognized the hazard and it may not be possible to correctly determine whether or not the examinee has actually recognized the hazard. Further, in the above-described conventional technology, the compensation action for compensating the visual field described above may not be appropriately reflected in the result of the risk assessment. Therefore, there is a problem that the conventional technology cannot appropriately assess the driving risk of the examinee.

Such a problem is not limited to the case of assessing the risk of driving an automobile, but is also common to the case of assessing the risk of driving/riding another type of vehicle (bicycles, for example) and the case of assessing a risk of moving on foot.

### SUMMARY

This specification discloses a technology capable of solving the above problems.

The technology disclosed herein may be implemented, for example, in the following forms.

(1) An information processing apparatus disclosed herein is an information processing apparatus for assessing a risk of moving of an examinee, including: a head information acquiring unit for acquiring head information for specifying movement of the head of the examinee; a display controlling unit for causing an image display device to display a simulated moving image that is a moving image simulating a view of a person moving on a predetermined course, includes a scene including a target object, and changes according to the movement of the head of the examinee specified by the head information; a viewpoint information acquiring unit for acquiring viewpoint information for specifying the position of the viewpoint of the examinee on the simulated moving image while the simulated moving image is displayed; and a risk assessing unit for assessing the risk on the basis of the degree of coincidence between the position of the viewpoint of the examinee specified by the viewpoint information and the position of the target object at the timing when the scene including the target object in the simulated moving image is displayed and outputting assessment information indicating the result of the assessment of the risk. Since the present information processing apparatus includes the head information acquiring unit for acquiring head information for specifying movement of the head of the examinee and the display controlling unit for causing an image display device to display a simulated moving image which changes according to the movement of the head of the examinee specified by the head information, the examinee can experience a natural moving situation in a simulated manner. The present information processing apparatus is also provided with a viewpoint information acquiring unit for acquiring viewpoint information for specifying the position of the viewpoint of the examinee on the simulated moving image while the simulated moving image is displayed, and a risk assessing unit for assessing the risk on the basis of the degree of coincidence between the position of the viewpoint of the examinee specified by the viewpoint information and the position of the target object at the timing when the scene including the target object in the simulated moving image is displayed and outputting assessment information indicating the result of the assessment of the risk. Therefore, according to the present information processing apparatus, it is possible to correctly determine whether or not the examinee has actually recognized the target object. Furthermore, according to the present information processing apparatus, even for an examinee who has a narrowed or defective visual field, such as an elderly person or a person who has developed an eye disease such as glaucoma, the risk of moving can be appropriately assessed by reflecting a compensation action for compensating the visual field by appropriately moving the head and eyes. Therefore, according to the present information processing apparatus, the risk of moving of the examinee can be appropriately assessed.

(2) The information processing apparatus may further include a visual field information acquiring unit for acquiring visual field information for specifying the visual field of the examinee, and the risk assessing unit may be configured to assess the risk on the basis of the degree of coincidence in the visual field of the examinee on the simulated moving image specified by the visual field information. According to the present information processing apparatus, it is possible to correctly determine whether or not the examinee has actually recognized the target object within the visual field even for an examinee whose visual field is narrowed or defective, such as an elderly person or a person who has developed an eye disease such as glaucoma. Therefore, according to the present information processing apparatus, even for an examinee whose visual field is narrowed or defective, the risk of moving of the examinee can be appropriately assessed.

(3) The information processing apparatus may further include an answer acquiring unit for acquiring an answer from the examinee while the simulated moving image is displayed, and the risk assessing unit may be configured to assess the risk on the basis of the degree of coincidence at the timing when the answer is acquired. According to the present information processing apparatus, when the viewpoint of the examinee coincides with the target object but the examinee does not recognize it as the target object, it can be correctly determined that the examinee did not recognize the target object, and the risk of moving of the examinee can be more appropriately assessed.

(4) In the information processing apparatus, the risk assessing unit may be configured to assess the risk on the basis of the degree of coincidence at a timing when the frequency in which the position of the viewpoint of the examinee specified by the viewpoint information is located within a region of a predetermined size in the simulated moving image within a predetermined time reaches or exceeds a predetermined threshold. When the frequency at which the viewpoint of the examinee is located within the region of the predetermined size in the simulated moving image within a predetermined time period reaches or exceeds a predetermined threshold value, it is highly likely that the examinee is gazing at something (what is drawn in the region) in the simulated moving image. Therefore, according to the present information processing apparatus, it is possible to determine (estimate) that the examinee has recognized the target obj ect in the simulated moving image without depending on a method such as an operation or an oral method and to appropriately assess a risk of moving of the examinee with a simpler configuration and a simpler method.

(5) In the information processing apparatus, the viewpoint information acquiring unit may be configured to acquire the viewpoint information for each of the right eye and the left eye individually, and the risk assessing unit may assess the risk on the basis of the degree of coincidence of at least one of the right eye and the left eye. According to the present information processing apparatus, the position of the viewpoint of the examinee can be more accurately specified, and the degree of coincidence between the position of the viewpoint of the examinee and the position of the target object can be more accurately determined. Therefore, according to the present information processing apparatus, it is possible to assess the risk of moving of the examinee more appropriately.

(6) The information processing apparatus may further include a dominant eye information acquiring unit for acquiring dominant eye information for specifying the dominant eye of the examinee, and the risk assessing unit may be configured to assess the risk on the basis of the degree of coincidence of the dominant eye of the examinee specified by the dominant eye information. According to the present information processing apparatus, it is possible to determine whether or not the examinee has visually recognized the target object with the dominant eye. Therefore, according to the present information processing apparatus, the risk of moving of the examinee can be further appropriately assessed.

(7) In the information processing apparatus, the simulated moving image may be a moving image that simulates a view of a person moving on the course while driving a vehicle. According to the present information processing apparatus, it is possible to appropriately assess a risk of moving of an examinee driving a vehicle.

(8) An information processing system disclosed herein may be configured to include the above-described information processing apparatus and the image display device. According to the present information processing system, it is possible to provide a system capable of appropriately assessing a risk of moving of an examinee while causing the examinee to view a simulated moving image.

(9) In the information processing system, the simulated moving image may be composed of a right eye image and a left eye image, and the image display device may be configured as a head-mounted display including a right eye display executing unit for causing the right eye of the examinee to view the right eye image and a left eye display executing unit, provided independently of the right eye display executing unit, for causing the left eye of the examinee to view the left eye image. According to the present information processing system, it is possible to cause the examinee to view the simulated moving image as a 3D image, to place the examinee in an environment very close to an actual moving environment, and to assess the risk of moving of the examinee more appropriately.

The technology disclosed herein can be implemented in various forms, for example, in the forms of an information processing apparatus, an information processing system, an information processing method, a computer program for implementing the method, and a non-temporary recording medium for storing the computer program, among other forms.

### BRIEF DESCRIPTION OF THE DRAWINGS

FIG. 1 is an explanatory diagram illustrating a schematic configuration of an information processing system 10 according to a first embodiment.

FIG. 2 is a block diagram illustrating a schematic configuration of the information processing system 10 according to the first embodiment.

FIG. 3 is a flowchart showing the contents of the driving risk assessment process in the first embodiment.

FIG. 4 is an explanatory diagram schematically illustrating a state of the examinee EX during the driving risk assessment process in the first embodiment and a simulated driving image SI viewed by the examinee EX.

FIG. 5 is an explanatory diagram schematically illustrating a state of the examinee EX during the driving risk assessment process in the first embodiment and a simulated driving image SI viewed by the examinee EX.

FIG. 6 is an explanatory diagram illustrating an example of a state in which assessment information ASI indicating a result of the driving risk assessment process in the first embodiment is displayed on the display unit 152. FIG. 7 is a block diagram illustrating a schematic configuration of an information processing system 10 according to a second embodiment.

FIG. 8 is a flowchart showing the contents of the driving risk assessment process in the second embodiment.

FIG. 9 is an explanatory diagram schematically illustrating a state of the examinee EX during the driving risk assessment process in the second embodiment and a simulated driving image SI viewed by the examinee EX.

FIG. 10 is an explanatory diagram illustrating an example of a state in which assessment information ASI indicating a result of the driving risk assessment process in the second embodiment is displayed on the display unit 152.

### DETAILED DESCRIPTION

A. First Embodiment:

It is useful to appropriately assess risks of drivers when driving an automobile and moving on a road. For example, when a driver's license is to be issued or renewed, such a driving risk assessment is performed to judge whether or not the driver's license can be issued or renewed on the basis of the result of the risk assessment, and appropriate education and training are conducted on the basis of the result of the risk assessment, resulting in reduction of the average risk of drivers and prevention of traffic accidents. In particular, elderly people and people having developed eye diseases such as glaucoma tend to have higher risks of driving because they may have narrowed or defective visual fields, while it is also possible to compensate for narrowed or defective visual fields by appropriately moving their head and eyes, and it is very useful to assess the driving risk in consideration of such compensation action. Examples of using the technology disclosed herein to properly assess a driving risk of a driver (examples applied to an information processing system 10) will be explained. The object of the assessment performed by the information processing system 10 is not the environmental risk as such, but is the examinee EX feeling the environmental risk.

A-1. Configuration of information processing system 10:

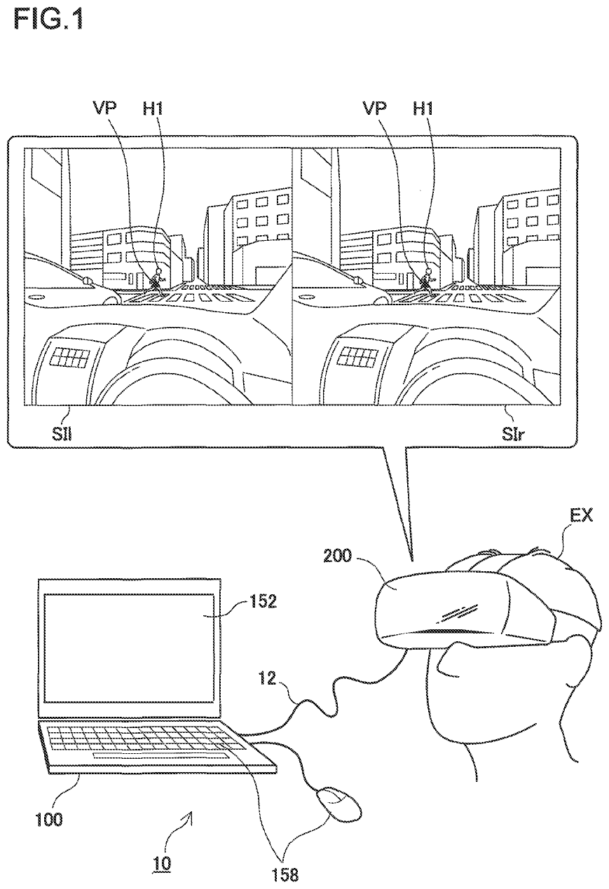

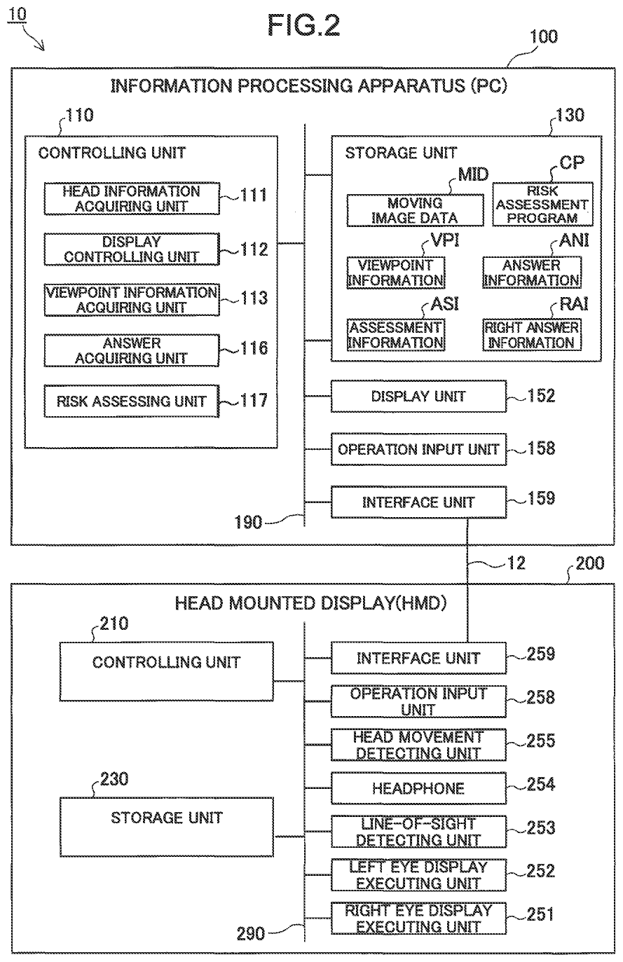

FIG. 1 is an explanatory diagram illustrating a schematic configuration of an information processing system 10 according to a first embodiment, and FIG. 2 is a block diagram illustrating a schematic configuration of the information processing system 10 according to the first embodiment. The information processing system 10 of this embodiment is a system for assessing a risk of the examinee EX when driving an automobile and moving on a road. More specifically, the information processing system 10 is a system for causing an examinee EX to view a simulated driving image SI (right eye image SIr and left eye image SI1) that simulates a human visual field moving on a predetermined course while driving an automobile, conducting a hazard recognition test to determine whether or not the examinee EX has recognized each hazard included in the simulated driving image SI, and assessing the risk of the examinee EX when driving an automobile and moving on a road on the basis of the result of the hazard recognition test.

As shown in FIGS. 1 and 2, the information processing system 10 includes a personal computer (hereinafter referred to as “PC”) 100 serving as an information processing apparatus and a head-mounted image display device (Head Mounted Display: hereinafter referred to as “HMD”) 200 serving as an image display device.

(Configuration of PC 100)

The PC 100 serving as an information processing apparatus includes a controlling unit 110, a storage unit 130, a display unit 152, an operation input unit 158, and an interface unit 159. These units are communicatively connected to each other via a bus 190.

The display unit 152 of the PC 100 is constituted by, for example, a liquid crystal display device and displays various images and information. The operation input unit 158 of the PC 100 is constituted by, for example, a keyboard, a mouse, and/or a microphone and receives operations and instructions from the operator and the examinee EX. The interface unit 159 of the PC 100 is constituted by, for example, a LAN interface or a USB interface and communicates with other devices through wired or wireless connection. In this embodiment, the interface unit 159 of the PC 100 is connected to the interface unit 259 of the HMD 200 (see below) via a cable 12 and communicates with the interface unit 259 of the HMD 200.

The storage unit 130 of the PC 100 is constituted by, for example, a ROM, a RAM, and/or a hard disk drive (HDD), stores various programs and data and is also used as a work area and a temporary data storage area when executing various programs. For example, the storage unit 130 stores a risk assessment program CP, which is a computer program for executing a driving risk assessment process described later. The risk assessment program CP is provided, for example, in a state of being stored in a computer-readable recording medium (not shown) such as a CD-ROM, a DVD-ROM, or a USB memory and is installed in the PC 100 to be stored in the storage unit 130.

The storage unit 130 of the PC 100 stores moving image data MID. The moving image data MID is data representing the simulated driving image SI described above.

The simulated driving image SI is a moving image having a predetermined frame rate (for example, 70 fps) and a predetermined length (for example, one minute). The simulated driving image SI is a moving image which simulates a human visual field moving on a predetermined course while driving an automobile and includes a plurality of scenes including a hazard Hn (n=1, 2, . . . ). The hazard Hn is, for example, an automobile, a bicycle, or a pedestrian, among others. For example, in a scene in the simulated driving image SI, a pedestrian as a hazard H1 jumps out from the side of a road. In this specification, the scene in the simulated driving image SI may be an image represented by one frame constituting the simulated driving image SI or an image having a predetermined time length represented by a plurality of consecutive frames.

The number of hazards Hn included in one scene may be one or plural. The simulated driving image SI is an example of a simulated moving image in the claims.

Further, in the present embodiment, as described later, since the simulated driving image SI to be viewed by the examinee EX is changed in accordance with movement of the head of the examinee EX, the moving image data MID includes data of a plurality of simulated driving images SI corresponding to each direction of the head of the examinee EX. Further, in the present embodiment, the simulated driving image SI is composed of a right eye image SIr and a left eye image SE created in consideration of parallax so that the simulated driving image SI to be viewed by the examinee EX is a 3D image. The moving image data MID representing the simulated driving image SI may be generated by, for example, 3D-CG software or may be generated by using an image captured by an omnidirectional camera mounted on an automobile traveling on an actual road. Further, the moving image data MID may include audio data representing a sound imitating noise of moving an automobile or the like.

The storage unit 130 of the PC 100 stores right answer information RAI. The right answer information RAI is information for specifying the timing at which a scene including each hazard Hn is displayed (display time point of the frame containing each hazard Hn) and the position of each hazard Hn in the simulated driving image SI (coordinates of the image region representing the hazard Hn on the frame). In addition, the storage unit 130 of the PC 100 stores viewpoint information VPI, answer information ANI, and assessment information ASI in the driving risk assessment process described later. The contents of these pieces of information will be described in conjunction with the description of the driving risk assessment process to be described later.

The controlling unit 110 of the PC 100 is constituted by, for example, a CPU and controls the operation of the PC 100 by executing a computer program read from the storage unit 130. For example, the controlling unit 110 reads the risk assessment program CP from the storage unit 130 and executes it, thereby executing the driving risk assessment process described later. More specifically, the controlling unit 110 functions as a head information acquiring unit 111, a display controlling unit 112, a viewpoint information acquiring unit 113, an answer acquiring unit 116, and a risk assessing unit 117 for executing a driving risk assessment process to be described later.

The functions of these components will be described in conjunction with the description of the driving risk assessment process described later.

(Configuration of HMD 200)

The HMD 200 served as an image display device is a device for causing the examinee EX to view an image while being mounted on the head of the examinee EX.

The HMD 200 of this embodiment is a non-transmissive head-mounted display that completely covers both eyes of the examinee EX and can provide a virtual reality (VR) function. In this specification, causing the examinee EX to view an image by the HMD 200 is also expressed as displaying an image (to the examinee EX) by the HMD 200.

The HMD 200 includes a controlling unit 210, a storage unit 230, a right eye display executing unit 251, a left eye display executing unit 252, a line-of-sight detecting unit 253, a headphone 254, a head movement detecting unit 255, an operation input unit 258, and an interface unit 259. These units are communicatively connected to each other via a bus 290.

The right eye display executing unit 251 of the HMD 200 includes, for example, a light source, a display element (digital mirror devices (DMD), liquid crystal panels, and the like), and an optical system, generates image light representing a right eye image SIr constituting the simulated driving image SI, and guides the image light to the right eye of the examinee EX, thereby causing the right eye of the examinee EX to view the right eye image SIr. The left eye display executing unit 252 is provided independently of the right eye display executing unit 251, and similarly to the right eye display executing unit 251, includes, for example, a light source, a display element, and an optical system, generates image light representing a left eye image SI1 constituting the simulated driving image SI, and guides the generated image light to the left eye of the examinee EX, thereby causing the left eye of the examinee EX to view the left eye image SE. In the state in which the right eye of the examinee EX views the right eye image SIr and the left eye of the examinee EX views the left eye image SE, the examinee EX views the simulated driving image SI as a 3D image.

The line-of-sight detecting unit 253 of the HID 200 detects the line-of-sight of the examinee EX in order to implement a so-called eye tracking function. For example, the line-of-sight detecting unit 253 includes a light source for emitting non-visible light and a camera, emits non-visible light from the light source, images the non-visible light reflected by the eye of the examinee EX by the camera to generate an image, and analyzes the generated image to detect the line-of-sight direction of the examinee EX. The line-of-sight detecting unit 253 repeatedly executes detection of the line-of-sight direction at a predetermined frequency (for example, at a frequency corresponding the frame rate of the moving image displayed by the right eye display executing unit 251 and the left eye display executing unit 252). It should be noted that the line-of-sight detecting unit 253 can specify the position of the viewpoint VP (see FIG. 1) of the examinee EX on the image that the examinee EX is viewing by detecting the line-of-sight of the examinee EX.

The headphone 254 of the HMD 200 is a device which is attached to the ears of the examinee EX and outputs sound. The head movement detecting unit 255 of the

HMD 200 is a sensor for detecting movement of the HMD 200 (that is, the movement of the head the examinee EX) to implement a so-called head tracking function. The movement of the head of the examinee EX is a concept including a change in the position and direction of the head of the examinee EX. The operation input unit 258 of the HMD 200 includes, for example, a button for receiving instructions from the examinee EX. The operation input unit 258 may be disposed inside the housing (the part mounted on the head of the examinee EX) of the HMD 200 or may be configured as a separate component connected to the housing via a signal line. The interface unit 259 of the HMD 200 includes, for example, a LAN interface or a USB interface and communicates with other devices through wired or wireless connection.

The storage unit 230 of the HMD 200 is constituted by, for example, a ROM and a RAM, stores various programs and data, and is used as a work area and a temporary data storage area when executing various programs. The controlling unit 210 of the HMD 200 is constituted by, for example, a CPU and controls the operation of each unit of the HMD 200 by executing a computer program read from the storage unit 230.

A-2. Driving Risk Assessment Process:

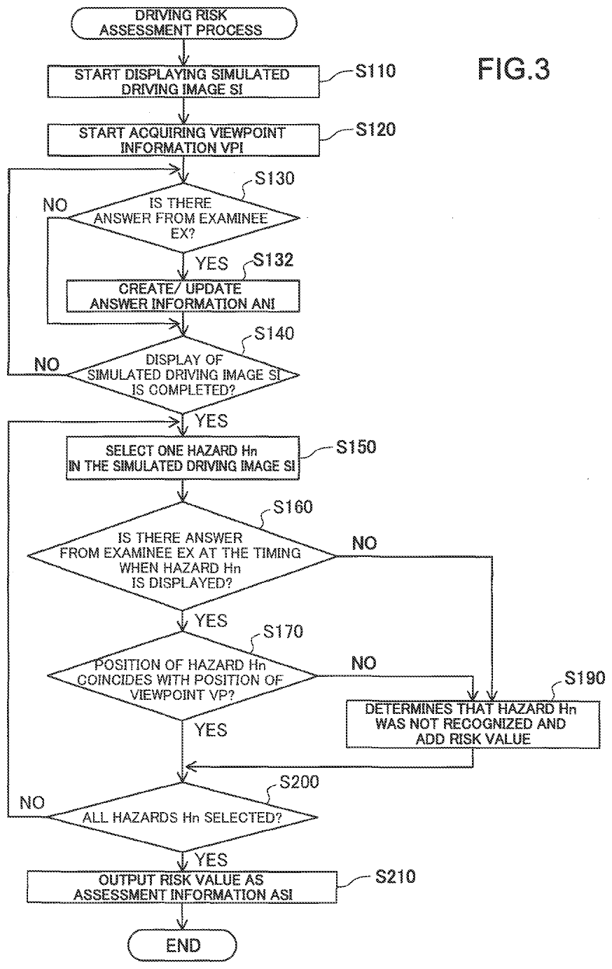

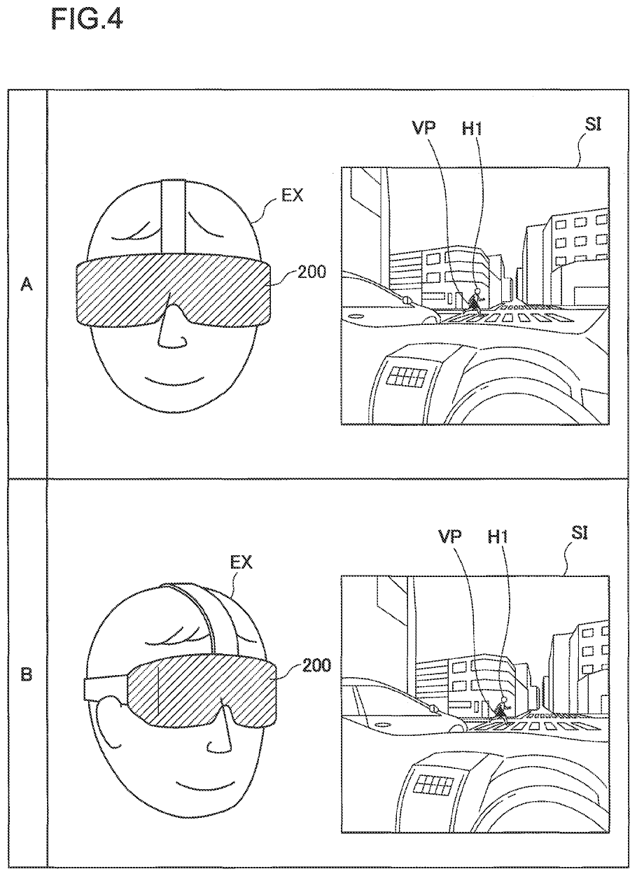

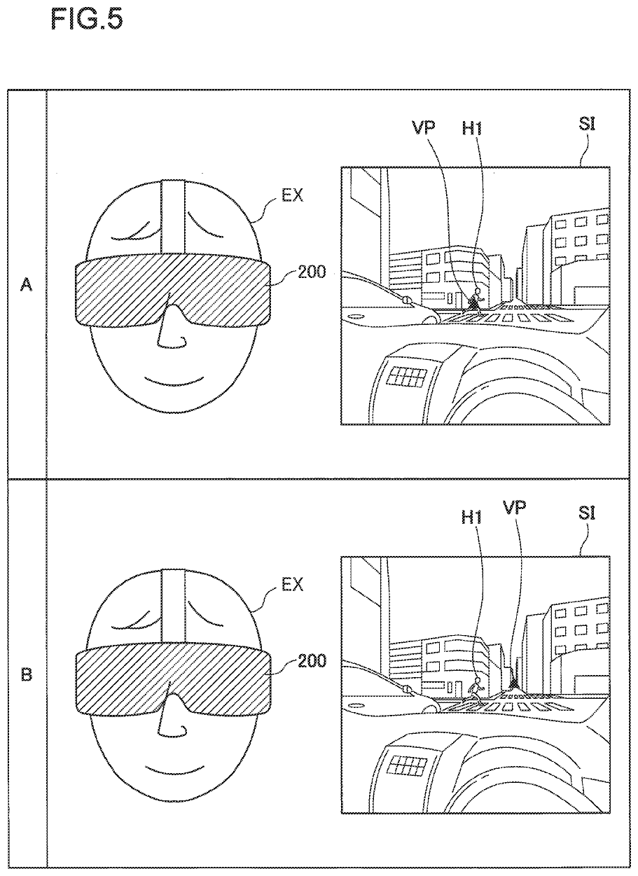

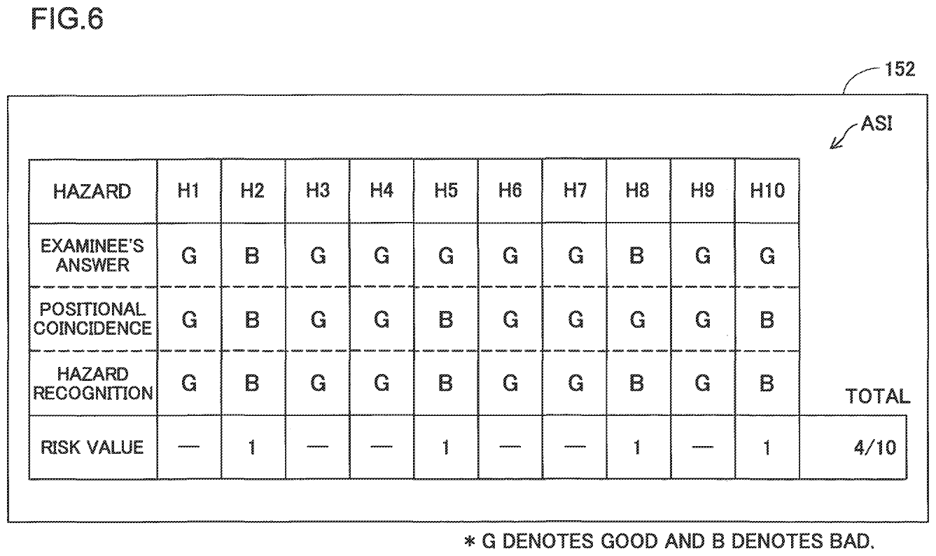

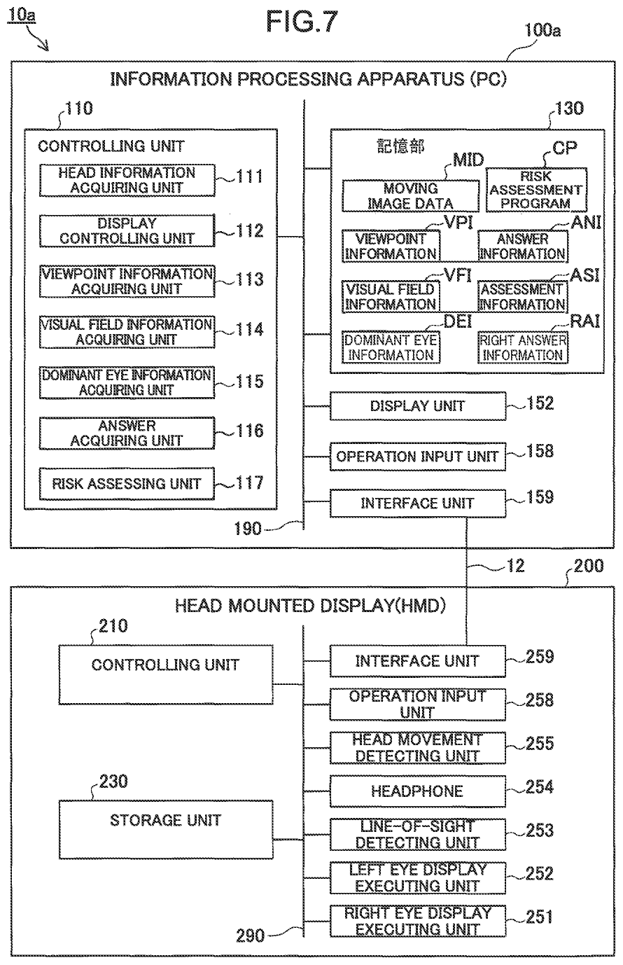

Next, the driving risk assessment process executed by the information processing system 10 of this embodiment will be described. FIG. 3 is a flowchart showing the contents of the driving risk assessment process in the first embodiment. FIGS. 4 and 5 are explanatory diagrams schematically illustrating states of the examinee EX at the time of the driving risk assessment processing in the first embodiment and the simulated driving image SI viewed by the examinee EX. FIG. 6 is an explanatory diagram illustrating an example of a state in which assessment information ASI indicating the result of the driving risk assessment process in the first embodiment is displayed on the display unit 152.

The driving risk assessment process is a process of causing the examinee EX to view the simulated driving image SI, conducting a hazard recognition test to determine whether or not the examinee EX has correctly recognized each hazard Hn included in the simulated driving image SI, and assessing the risk of the examinee EX when driving an automobile and moving on a road on the basis of the results of the hazard recognition test. In the hazard recognition test, the examinee EX is instructed to answer by the operation of the operation input unit 158 (for example, clicking the mouse) when the examinee EX recognizes something that the examinee EX considers as a hazard Hn. For example, in a state in which the examinee EX wears the HMD 200, the driving risk assessment process is started in response to an instruction for starting the process input by an operator via the operation input unit 158 of the PC 100.

When the driving risk assessment process is started, the display controlling unit 112 of the PC 100 causes the HMD 200 to start displaying the simulated driving image SI (S110). More specifically, the display controlling unit 112 of the PC 100 supplies the moving image data MID stored in the storage unit 130 to the HMD 200 and causes the right eye display executing unit 251 and the left eye display executing unit 252 of the HMD 200 to display the right eye image SIr and the left eye image SE constituting the simulated driving image SI, respectively.

It should be noted that the information processing system 10 of the present embodiment has a so-called head tracking function and changes the simulated driving image SI viewed by the examinee EX according to the movement of the head of the examinee EX. That is, the head information acquiring unit 111 of the PC 100 acquires head information specifying the head movement of the examinee EX detected by the head movement detecting unit 255 of the HMD 200 from the HMD 200, and the display controlling unit 112 of the PC 100 selects the moving image data MID supplied to the right eye display executing unit 251 and the left eye display executing unit 252 of the HMD 200 according to the head movement of the examinee EX specified by the acquired head information. Thus, the examinee EX views the simulated driving image SI which naturally changes according to the movement of the examinee's own head. For example, when the examinee EX changes the direction of the head from a state where the examinee EX faces the front and sees an image of a scene as shown in the column A of FIG. 4 to the left as shown in the column B of FIG. 4, the image viewed by the examinee EX naturally changes to an image of a scene shifted to the left from the scene. Thus, the examinee EX is placed in an environment very close to an actual driving environment in terms of vision. The selection of the moving image data MID supplied to the right eye display executing unit 251 and the left eye display executing unit 252 may be executed by the controlling unit 210 of the HMD 200.

Simultaneously with the start of display of the simulated driving image SI, the viewpoint information acquiring unit 113 of the PC 100 starts a process of acquiring, from the HMD 200, viewpoint information VPI for specifying the position of the viewpoint VP of the examinee EX on the simulated driving image SI specified by the line-of-sight detecting unit 253 of the HMD 200 (S120). The viewpoint information VPI is information for specifying the position (coordinates) of the viewpoint VP of the examinee EX at each time point of the simulated driving image SI. By referring to the viewpoint information VPI, it is possible to grasp where the examinee EX is gazing at in each scene of the simulated driving image SI. The acquired viewpoint information VPI is stored in the storage unit 130. It should be noted that, in FIGS. 1, 4, and 5, for example, a mark indicating the viewpoint VP is drawn over the simulated driving image SI for convenience of explanation, but in this embodiment, the mark indicating the viewpoint VP is not actually displayed as an image so that the examinee EX under test is not conscious of the position of his/her own viewpoint VP. However, a mark indicating the viewpoint VP may be displayed (may be visible to the examinee EX).

While the simulated driving image SI is displayed, the answer acquiring unit 116 of the PC 100 monitors whether or not there is an answer (operation of the operation input unit 158) from the examinee EX (S130), and if it is determined that there is an answer (S130: YES), creates and updates the answer information ANI (S132). The answer information ANI is information for specifying a time point (time point in the simulated driving image SI) at which an answer was made by the examinee EX. By referring to the answer information ANI, it is possible to grasp at what time (that is, in which scene) in the simulated driving image SI the examinee EX recognized something that the examinee considered to be a hazard Hn. The created/updated answer information ANI is stored in the storage unit 130. If it is determined in S130 that there is no answer (S130: NO), the process in S132 is skipped.

The display controlling unit 112 of the PC 100 monitors whether or not the display of the simulated driving image SI is completed (S140). If it is determined in S140 that the display of the simulated driving image SI has not been completed (S140: NO), the processes in and after S130 are repeatedly executed. If it is determined in S140 that the display of the simulated driving image SI has been completed (S140: YES), it means that the hazard recognition test for the examinee EX has been completed, and the process proceeds to S150.

When the hazard recognition test for the examinee EX is completed, the risk assessing unit 117 of the PC 100 refers to the right answer information RAI previously stored in the storage unit 130, as well as the answer information ANI and the viewpoint information VPI created and updated during the test, thereby starting the driving risk assessment of the examinee EX as described in detail below.

First, the risk assessing unit 117 of the PC 100 selects one hazard Hn in the simulated driving image SI (S150) and refers to the right answer information RAI and the answer information ANI to determine whether or not there is an answer from the examinee EX at the timing when the scene including the selected hazard Hn is displayed (S160). In S160, if it is determined that there is no answer from the examinee EX at the timing when the scene including the hazard Hn is displayed (S160: NO), the risk assessing unit 117 determines that the examinee EX could not recognize the hazard Hn and adds a risk value (S190). In the example of the assessment result shown in FIG. 6, since the examinee EX did not answer at the timing when the scene including the hazard H2 or the hazard H8 was displayed (examinee's answer: BAD (hereinafter, denoted as “B”)), it is determined that the examinee EX could not recognize these hazards (hazard recognition: B), and the risk value “1” is added.

On the contrary, if it is determined in S160 that the examinee EX has answered at the timing when the scene including the selected hazard Hn is displayed (S160: YES), the risk assessing unit 117 of the PC 100 refers to the right answer information RAI and the viewpoint information VPI to determine whether or not the position of the hazard Hn in the scene including the selected hazard Hn coincides with the position of the viewpoint VP of the examinee EX at the timing when the scene including the hazard Hn is displayed (S170). In this specification, coincidence of the position of the hazard Hn with the position of the viewpoint VP of the examinee EX means that the degree of coincidence between the positions is equal to or higher than a predetermined threshold value. That is, the risk assessing unit 117 determines that the position of the hazard Hn coincides with the position of the viewpoint VP of the examinee EX when the ratio of the length of time during which the position of the viewpoint VP of the examinee EX coincides with the position (region) of the hazard Hn to the length of time during which the scene including the hazard Hn is displayed (that is, the degree of coincidence between the position of the hazard Hn and the position of the viewpoint VP of the examinee EX) is equal to or higher than a predetermined threshold value. The column A in FIG. 5 shows an example in which the position of the hazard Hn coincides with the position of the viewpoint VP of the examinee EX, and the column B in FIG. 5 shows an example in which the position of the hazard Hn does not coincide with the position of the viewpoint VP of the examinee EX.

When it is determined in S170 that the position of the hazard Hn does not coincide with the position of the viewpoint VP of the examinee EX (S170: NO), the risk assessing unit 117 determines that the examinee EX could not actually recognize the hazard Hn although the examinee EX answered at the timing when the scene including the hazard Hn is displayed (that is, the examinee EX mistakenly recognized another object in the scene as a hazard, or the answer by the examinee EX was an erroneous operation) and adds the risk value (S190). In the example of the assessment result shown in FIG. 6, with regard to hazard H5 and hazard H10, since the position of hazard Hn did not coincide with the position of the viewpoint VP of examinee EX (positional coincidence: B), it is determined that examinee EX could not recognize these hazards (hazard recognition: B) so that the risk value “1” is added.

On the contrary, if it is determined in S170 that the position of the hazard Hn coincides with the position of the viewpoint VP of the examinee EX (S170: YES), the risk assessing unit 117 determines that the examinee EX has correctly recognized the hazard Hn, does not perform the risk value addition processing (S190), and advances the processing to S200.

Upon completion of the determination as to whether or not the examinee EX could recognize the hazard Hn for the selected one hazard Hn (S160 to S190), the risk assessing unit 117 determines whether or not all the hazards Hn included in the simulated driving image SI have been selected (S200), and if it is determined that there is an unselected hazard Hn (S200: NO), the process returns to the selection process of the hazard Hn (S150), and the subsequent processes are performed in the same manner. In the example of the assessment result shown in FIG. 6, since there are ten hazards Hn, the determination as to whether or not the examinee EX could recognize the hazard Hn is repeatedly executed for each hazard Hn until it is determined that the selection of ten hazards Hn has been completed.

After repeating these steps, when it is determined in 5200 that all the hazards Hn have been selected (S200: YES), the risk assessing unit 117 generates assessment information ASI representing the result of the risk assessment (for example, the sum of the risk values) and outputs the assessment information ASI (S210). For example, as shown in FIG. 6, the risk assessing unit 117 displays the contents of the assessment information ASI on the display unit 152. Thus, the driving risk assessment process of assessing the risk of the examinee EX when driving an automobile and moving on a road is completed. In the example of the assessment result shown in FIG. 6, because the examinee EX failed to recognize four hazards (H2, H5, H8, and H10) of the ten hazards Hn, the total of the risk values was 4 points (with the maximum risk value being 10 points). The higher the risk value, the higher the risk of examinee EX when driving an automobile on a road.

A-3. Effect of First Embodiment:

As described above, the PC 100 constituting the information processing system 10 of the first embodiment is an information processing apparatus for assessing a risk of the examinee EX when moving by driving an automobile and includes the head information acquiring unit 111, the display controlling unit 112, the viewpoint information acquiring unit 113, and the risk assessing unit 117. The head information acquiring unit 111 acquires head information specifying movement of the head of the examinee EX. The display controlling unit 112 causes the HMD 200 as an image display device to display the simulated driving image SI which is a moving image simulating the visual field of a human who drives an automobile and moves on a predetermined course. The simulated driving image SI includes a scene including the hazard Hn and is a moving image which changes according to the movement of the head of the examinee EX specified by the head information. The viewpoint information acquiring unit 113 acquires viewpoint information VPI for specifying the position of the viewpoint VP of the examinee EX on the simulated driving image SI while the simulated driving image SI is displayed. The risk assessing unit 117 assesses a risk of the examinee EX when moving by driving an automobile on the basis of the degree of coincidence between the position of the viewpoint VP of the examinee EX specified by the viewpoint information VPI and the position of the hazard Hn at the timing when a scene including the hazard Hn in the simulated driving image SI is displayed and outputs assessment information ASI indicating the result of the assessment of the risk.

Thus, the PC 100 of the present embodiment includes the head information acquiring unit 111 for acquiring head information specifying movement of the head of the examinee EX and the display controlling unit 112 for causing the HMD 200 to display a simulated driving image SI that changes in accordance with the movement of the head of the examinee EX specified by the head information, so that the examinee

EX can experience a natural driving situation in a simulated manner. For example, in a scene of an intersection with poor visibility, the movement of the head and the eyes is important for checking right and left, and according to the PC 100 of the present embodiment, since the simulated driving image SI viewed by the examinee EX changes in accordance with the head movements of the examinee EX, the rotation of the head and the rotation of the eyes can be made to be natural movements so that the examinee EX can experience a natural driving situation in a simulated manner.

The PC 100 of the present embodiment is also provided with the viewpoint information acquiring unit 113 for acquiring viewpoint information VPI for specifying the position of the viewpoint VP of the examinee EX on the simulated driving image

SI while the simulated driving image SI is displayed and the risk assessing unit 117 for assessing the risk on the basis of the degree of coincidence between the position of the viewpoint VP of the examinee EX specified by the viewpoint information VPI and the position of the hazard Hn at the timing when the scene including the hazard Hn in the simulated driving image SI is displayed and outputting assessment information ASI indicating the result of the assessment of the risk. Therefore, according to the PC 100 of the present embodiment, it is possible to correctly determine whether or not the examinee EX has actually recognized the hazard Hn. Furthermore, according to the PC 100 of the present embodiment, even for an examinee EX having a narrowed or defective visual field such as an elderly person or a person who has developed an eye disease such as glaucoma, the driving risk can be appropriately assessed by reflecting a compensation action for compensating the visual field by appropriately moving the head and eyes.

From the above, according to the PC 100 of the present embodiment, the driving risk of the examinee EX can be appropriately assessed.

The PC 100 of the present embodiment further includes the answer acquiring unit 116 for acquiring an answer from the examinee EX while the simulated driving image SI is displayed. Further, the risk assessing unit 117 of the PC 100 assesses the risk of the examinee EX when moving by driving an automobile on the basis of the degree of coincidence between the position of the viewpoint VP of the examinee EX specified by the viewpoint information VPI and the position of the hazard Hn at the timing when the answer by the examinee EX is acquired. Therefore, according to the PC 100 of the present embodiment, when the viewpoint VP of the examinee EX coincides with the hazard Hn but the examinee EX does not recognize it as the hazard Hn, it can be correctly determined that the examinee EX did not recognize the hazard Hn so that the driving risk of the examinee EX can be more appropriately assessed.

In addition, the information processing system 10 of this embodiment includes the PC 100 and the HMD 200. Therefore, according to the information processing system 10 of the present embodiment, it is possible to provide a system capable of appropriately assessing the driving risk of the examinee EX while causing the examinee EX to view the simulated driving image SI.

In the present embodiment, the simulated driving image SI is composed of the right eye image SIr and the left eye image SIl. In addition, the HMD 200 is a head-mounted display including a right eye display executing unit 251 for causing the right eye of the examinee EX to view the right eye image SIr, and a left eye display executing unit 252, provided independently of the right eye display executing unit 251, for causing the left eye of the examinee EX to view the left eye image SIl. Therefore, according to the information processing system 10 of the present embodiment, it is possible to cause the examinee EX to view the simulated driving image SI as a 3D image, to place the examinee EX in an environment very close to an actual driving environment, and to assess the driving risk of the examinee EX more appropriately.

B. Second Embodiment:

FIG. 7 is a block diagram illustrating a schematic configuration of an information processing system 10a according to a second embodiment. In the following, among the components of the information processing system 10a of the second embodiment and the processing contents performed by the information processing system 10a, the same components and processing contents as those of the above-described first embodiment are denoted by the same reference numerals, and the description thereof will be omitted as appropriate.

As shown in FIG. 7, the information processing system 10a of the second embodiment includes a PC 100a the configuration of which differs from that of the first embodiment. Specifically, in the information processing system 10a according to the second embodiment, the controlling unit 110 of the PC 100a reads a risk assessment program CP from the storage unit 130 and executes it so as to function as a visual field information acquiring unit 114 and a dominant eye information acquiring unit 115. The functions of these components will be described in conjunction with the description of the driving risk assessment process described later.

Further, in the information processing system 10a of the second embodiment, during the driving risk assessment process to be described later, visual field information VFI and dominant eye information DEI are further stored in the storage unit 130 of the PC 100a. The contents of these pieces of information will be described in conjunction with the description of the driving risk assessment process to be described later.

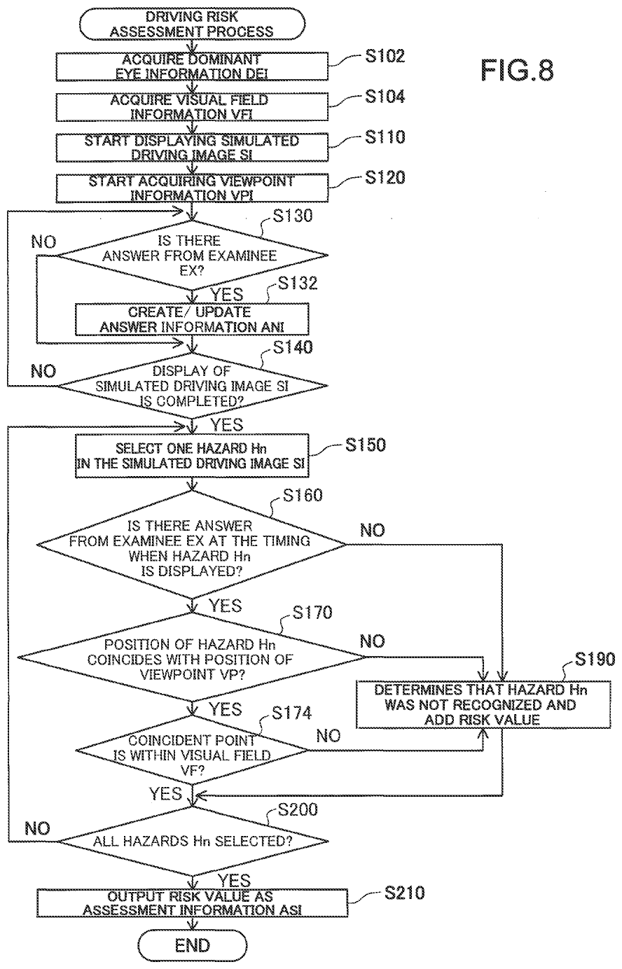

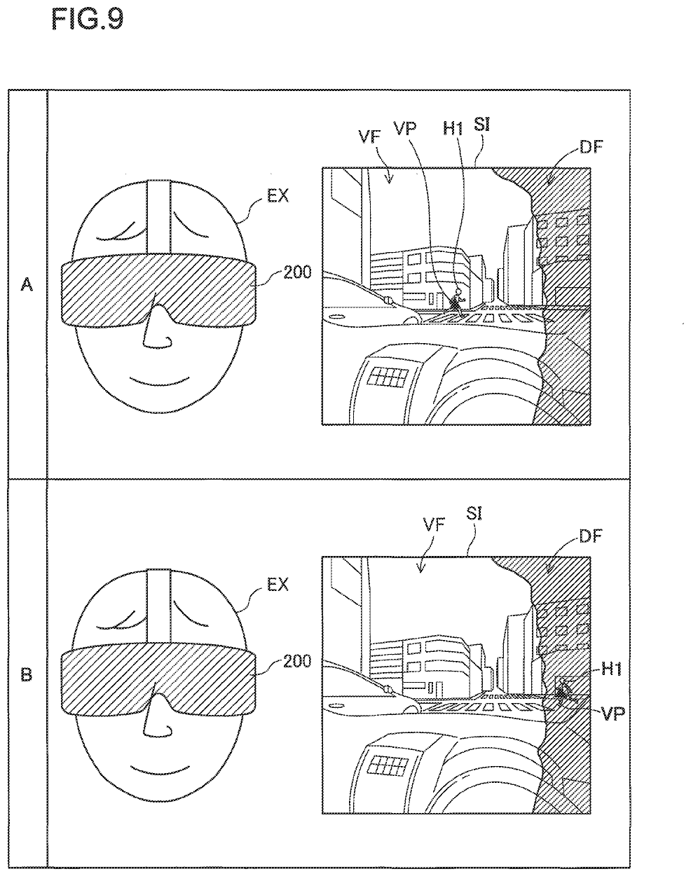

FIG. 8 is a flowchart showing the contents of the driving risk assessment process in the second embodiment. FIG. 9 is an explanatory diagram schematically illustrating a state of the examinee EX at the time of the driving risk assessment process in the second embodiment and the simulated driving image SI viewed by the examinee EX.

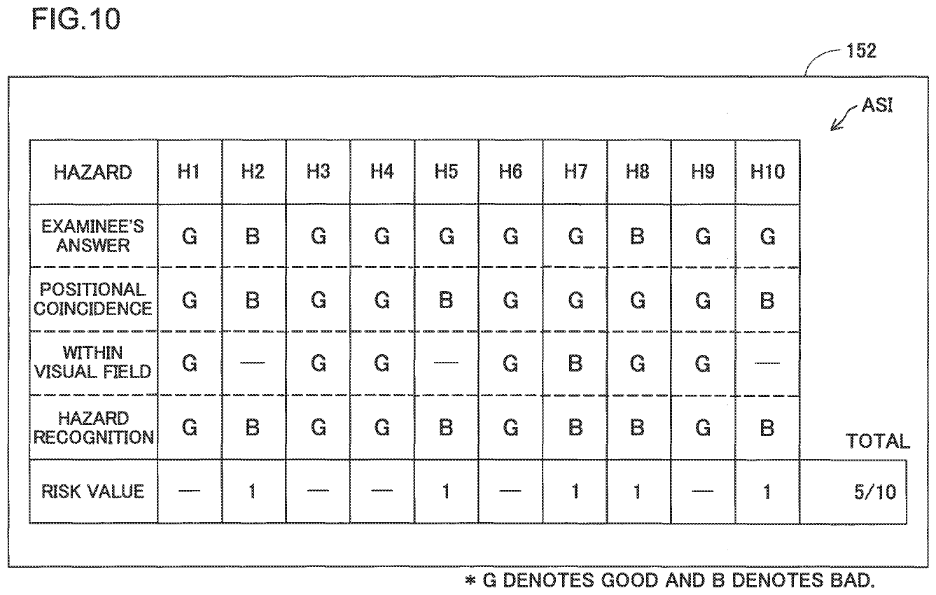

FIG. 10 is an explanatory diagram illustrating an example of a state in which assessment information ASI indicating the result of the driving risk assessment process in the second embodiment is displayed on the display unit 152.

In the driving risk assessment process of the second embodiment, the hazard recognition test is executed as in the first embodiment, but before the hazard recognition test is started, the dominant eye information acquiring unit 115 of the PC 100a acquires the dominant eye information DEI for specifying the dominant eye of the examinee EX (S102). The dominant eye information DEI may be acquired in accordance with information input from the operation input unit 158 (information to specify the dominant eye) or may be acquired on the basis of the result of a test for determining the dominant eye performed by the information processing system 10a. The dominant eye information DEI is stored in the storage unit 130. In the present embodiment, it is assumed that the dominant eye of the examinee EX is the right eye.

The visual field information acquiring unit 114 of the PC 100a acquires the visual field information VFI for specifying the visual field of the examinee EX (S104). The visual field information VFI may be acquired in accordance with information input from the operation input unit 158 (information identifying a visual field measured by a perimeter), or the information processing system 10a itself may include a perimeter and acquire the visual field on the basis of the result of measurement by the perimeter. The visual field information VFI is stored in the storage unit 130. In this embodiment, as shown in FIG. 9, it is assumed that a visual field defect DF exists in the visual field VF of the examinee EX, so that the visual field VF is narrower than the range in which the visual point VP could be located (the range in the viewing direction).

Thereafter, as in the first embodiment, a hazard recognition test (S110 to S140) for the examinee EX is started. The processing contents of the hazard recognition test in the second embodiment are basically the same as those in the first embodiment. However, in the second embodiment, in the process of acquiring the viewpoint information VPI (S120), the viewpoint information VPI is individually acquired for each of the right eye and the left eye of the examinee EX. That is, the line-of-sight detecting unit 253 of the HMD 200 detects each of the line-of-sight directions of the right eye and the left eye of the examinee EX, thereby specifying the positions of the respective viewpoints VP of the right eye and the left eye of the examinee EX. The viewpoint information acquiring unit 113 of the PC 100a acquires viewpoint information VPI for specifying the positions of the viewpoints VP of the right eye and the left eye of the examinee EX specified by the line-of-sight detecting unit 253 of the HMD 200 from the HMD 200.

When the hazard recognition test for the examinee EX is completed, a driving risk assessment for the examinee EX is started as in the first embodiment. The processing contents of the risk assessment in the second embodiment are basically the same as those in the first embodiment. However, the second embodiment is different from the first embodiment in that in the process of determining the degree of coincidence between the position of the hazard Hn and the position of the viewpoint VP of the examinee EX (S170), the degree of coincidence between the position of the hazard Hn and the position of the viewpoint VP of the dominant eye (right eye in this embodiment) of the examinee EX is determined.

Moreover, in the second embodiment, when it is determined that the position of the hazard Hn coincides with the position of the viewpoint VP of the dominant eye of the examinee EX (S170: YES), the risk assessing unit 117 further determines whether or not the coincident point is within the visual field VF of the examinee EX (S174). As shown in the column B of FIG. 9, when it is determined in S174 that the point of coincidence between the position of the hazard Hn and the position of the viewpoint

VP is not within the visual field VF of the examinee EX (that is, within the visual field defect DF) (S174: NO), the risk assessing unit 117 determines that the examinee EX answered at the timing when the scene including the hazard Hn is displayed and that the position of the hazard Hn coincides with the position of the viewpoint VP of the examinee EX, but the examinee EX could not actually recognize the hazard Hn because the point of coincidence was not within the visual field VF of the examinee EX, and adds the risk value (S190). In the example of the assessment result shown in FIG. 10, since the point of coincidence between the position of the hazard H7 and the position of the viewpoint VP was not within the visual field VF of the examinee EX (within visual field: B), it is determined that the examinee EX could not recognize this hazard (hazard recognition: B), and the risk value “1” is added.

On the contrary, as shown in column A of FIG. 9, when it is determined in S174 that the point of coincidence between the position of the hazard Hn and the position of the viewpoint VP is within the visual field VF of the examinee EX (S174: YES), the risk assessing unit 117 determines that the examinee EX has correctly recognized the hazard Hn, does not perform the risk value addition processing (S190), and advances the processing to 5200. The subsequent processing is the same as in the first embodiment. In the example of the assessment result shown in FIG. 10, because the examinee EX failed to recognize five hazards Hn (H2, H5, H7, H8, and H10) out of ten hazards Hn, the total of the risk value is 5 points (with the maximum risk value being 10 points).

As described above, since the PC 100a constituting the information processing system 10a of the second embodiment has the same configuration as that of the PC 100 of the first embodiment, the driving risk of the examinee EX can be appropriately assessed as in the PC 100 of the first embodiment.

Further, the PC 100a of the second embodiment includes the visual field information acquiring unit 114 for acquiring the visual field information VFI for specifying the visual field of the examinee EX. The risk assessing unit 117 assesses the risk of the examinee EX when moving by driving an automobile on the basis of the degree of coincidence between the position of the viewpoint VP of the examinee EX and the position of the hazard Hn within the visual field of the examinee EX on the simulated driving image SI specified by the visual field information VFI. Therefore, according to the PC 100a of the second embodiment, even for an examinee EX having a narrowed or defective visual field, such as an elderly person or a person who has developed an eye disease such as glaucoma, it is possible to correctly determine whether or not the examinee EX has actually recognized the hazard Hn in the visual field VF. Therefore, according to the PC 100a of the second embodiment, it is possible to appropriately assess the driving risk of the examinee EX even for an examinee EX whose visual field is narrowed or defective.

In the second embodiment, the viewpoint information acquiring unit 113 of the PC 100a acquires viewpoint information VPI for each of the right eye and the left eye of the examinee EX individually. In addition, the PC 100a of the second embodiment includes a dominant eye information acquiring unit 115 for acquiring dominant eye information DEI for specifying the dominant eye of the examinee EX. Moreover, in the second embodiment, the risk assessing unit 117 assesses the risk of the examinee EX when moving by driving an automobile on the basis of the degree of coincidence between the position of the viewpoint VP of the examinee EX and the position of the hazard Hn with respect to the dominant eye of the examinee EX identified by the dominant eye information DEI. Therefore, according to the PC 100a of the second embodiment, it is possible to determine whether or not the examinee EX has visually recognized the hazard Hn with the dominant eye. Therefore, according to the PC 100a of the second embodiment, the risk of the examinee EX during operation can be more appropriately assessed.

C. Modifications:

The technology disclosed herein are not limited to the embodiments described above and can be modified in various forms without departing from the scope of the invention, such as the following modifications.

The configuration of the information processing system 10 in the above-described embodiment is merely an example and can be variously modified. For example, in the above-described embodiment, the PC 100 is used as the information processing apparatus constituting the information processing system 10, but other types of computers (for example, smart phones or tablet devices) may be used as the information processing apparatus. Further, in the above-described embodiment, the HMD 200 is used as the image display device constituting the information processing system 10, but other types of image display devices (for example, liquid crystal displays or projectors) may be used as the image display device. It should be noted that when a device other than the HMD 200 (image display device not mounted on head) is used as the image display device, a sensor for detecting the direction of the line-of-sight of the examinee EX and a sensor for detecting the head movement of the examinee EX may be used separately from the image display device to detect the direction of the line-of-sight of the examinee EX and the head movement of the examinee EX.

The information processing apparatus and the image processing apparatus constituting the information processing system 10 may be an integrated apparatus. For example, the information processing system 10 may be composed of only the HMD 200 provided with the functions of the PC 100 of the above-described embodiment.

The contents of the driving risk assessment process in the above-described embodiment are merely examples and may be variously modified. For example, in the driving risk assessment process in the above-described embodiment, the simulated driving image SI includes a scene including the hazard Hn, and the driving risk is assessed by determining whether or not the examinee EX correctly recognized each hazard Hn, but the target object to be recognized by the examinee EX is not limited to the hazard Hn and may be an object that affects the driving risk, such as a traffic light or a road sign. That is, the simulated driving image SI may be a moving image that simulates a visual field of a person moving on a predetermined course and may include a scene including target objects, and the risk assessing unit 117 may assess a driving risk on the basis of the degree of coincidence between the position of the viewpoint VP of the examinee EX and the position of the target object at the timing when the scene including the target object in the simulated driving image SI is displayed.

Further, in the driving risk assessment process in the above-described embodiment, the risk assessing unit 117 determines that the position of the hazard Hn coincides with the position of the viewpoint VP of the examinee EX when the ratio of the length of time in which the position of the viewpoint VP of the examinee EX coincides with the position (region) of the hazard Hn to the length of time in which the scene including the hazard Hn is displayed (that is, the degree of coincidence between the position of the hazard Hn and the position of the viewpoint VP of the examinee EX) is equal to or higher than a predetermined threshold value, but the determination method of the degree of coincidence between the position of the hazard Hn and the position of the viewpoint VP of the examinee EX can be modified in various ways.

In the driving risk assessment process in the above-described embodiment, when the examinee EX recognizes each hazard Hn included in the simulated driving image SI in the hazard recognition test, the examinee EX answers by the operation of the operation input unit 158, but the method for the answer is not limited to the operation of the operation input unit 158 and may be another method. For example, the examinee EX may answer orally. Alternatively, the risk assessing unit 117 of the PC 100 may determine that there is an answer from the examinee EX when the position of the viewpoint VP of the examinee EX satisfies a specific condition. For example, when the frequency at which the viewpoint VP of the examinee EX specified by the viewpoint information VPI is located within a region of a predetermined size in the simulated driving image SI within a predetermined time reaches or exceeds a predetermined threshold, it may be determined that there is an answer from the examinee EX. If the frequency at which the viewpoint VP of the examinee EX is located within the region of the predetermined size in the simulated driving image SI within the predetermined time reaches or exceeds the predetermined threshold value, it is considered highly probable that the examinee EX is gazing at something (what is drawn in the above region) in the simulated driving image SI, and therefore, by adopting such a scheme, it can be determined (estimated) that the examinee EX has recognized the hazard Hn in the simulated driving image SI without depending on a method such as an operation of the operation input unit 158 or an oral method, and the driving risk of the examinee EX can be appropriately assessed by a simpler configuration and a simpler method.

Although the assessment information ASI is output by being displayed on the display unit 152 in the above-described embodiment, the output form of the assessment information ASI may be, for example, other forms such as an audio output or an output by printing using a printing device. In the above-described embodiment, the contents of the output assessment information ASI are merely examples and can be variously modified. For example, among the contents shown in FIG. 6, the assessment information ASI may not include the results of “examinee's answer” and “positional coincidence” but may include only the result of “hazard recognition” for each hazard

In Alternatively, the assessment information ASI may include only the final sum of the risk values (for example, 4/10 points), without including the content indicating the appropriateness of recognition of each hazard Hn shown in FIG. 6.

Further, in the driving risk assessment process in the above-described embodiment, whether or not there is an answer from the examinee EX is monitored during the hazard recognition test (S130), and whether or not there is an answer from the examinee EX at a timing when a scene including the hazard Hn is displayed during the subsequent risk assessment is determined (S160), but these steps may be omitted.

In the driving risk assessment process in the second embodiment, the dominant eye information DEI and the visual field information VFI are acquired and used, but only one of the dominant eye information DEI and the visual field information VFI may be acquired and used. For example, in the driving risk assessment process in the second embodiment, the visual field information VFI may be acquired but the dominant eye information DEI may not be acquired, and the degree of coincidence between the position of the hazard Hn and the position of the viewpoint VP of the examinee EX may be determined for both eyes.

In the above-described embodiment, the degree of coincidence between the position of the viewpoint VP of the examinee EX and the position of the hazard Hn may be determined only for the viewpoint VP of the right eye, only for the viewpoint VP of the left eye, or for the viewpoint VP of both eyes.

Further, in the above-described embodiment, the information processing system 10 assesses the risk of the examinee EX when driving an automobile and moving on a road, but the information processing system 10 may assess the risk of the examinee EX in moving by other means (for example, by driving/riding another type of vehicle (a bicycle, for example) or on foot). In these cases, instead of the simulated driving image SI in the embodiment described above, an image that simulates a human visual field moving by the other means on a predetermined course may be used.

Moreover, in each of the above-described embodiments, a part of the configuration implemented by a hardware may be replaced with a software, and on the contrary, a part of the configuration implemented by a software may be replaced with a hardware.

What is claimed is:

1. An information processing apparatus for assessing a risk of moving of an examinee, comprising:
a head information acquiring unit for acquiring head information for specifying movement of the head of the examinee;
a display controlling unit for causing an image display device to display a simulated moving image that is a moving image simulating a view of a person moving on a predetermined course, includes a scene including a target object, and changes according to the movement of the head of the examinee specified by the head information;
a viewpoint information acquiring unit for acquiring viewpoint information for specifying the position of the viewpoint of the examinee on the simulated moving image while the simulated moving image is displayed; and
a risk assessing unit for assessing the risk on the basis of the degree of coincidence between the position of the viewpoint of the examinee specified by the viewpoint information and the position of the target object at the timing when the scene including the target object in the simulated moving image is displayed and outputting assessment information indicating the result of the assessment of the risk.

2. The information processing apparatus according to claim 1, further comprising:
a visual field information acquiring unit for acquiring visual field information for specifying the visual field of the examinee,
wherein the risk assessing unit assesses the risk on the basis of the degree of coincidence in the visual field of the examinee on the simulated moving image specified by the visual field information.

3. The information processing apparatus according to claim 1, further comprising:
an answer acquiring unit for acquiring an answer from the examinee while the simulated moving image is displayed,
wherein the risk assessing unit assesses the risk on the basis of the degree of coincidence at the timing when the answer is acquired.

4. The information processing apparatus according to claim 1 or 2,
wherein the risk assessing unit assesses the risk on the basis of the degree of coincidence at the timing when the frequency in which the position of the viewpoint of the examinee specified by the viewpoint information is located within a region of a predetermined size on the simulated moving image within a predetermined time reaches or exceeds a predetermined threshold.

5. The information processing apparatus according to any one of claims 1 to 4,
wherein the viewpoint information acquiring unit acquires the viewpoint information for each of the right eye and the left eye individually, and
wherein the risk assessing unit assesses the risk on the basis of the degree of coincidence of at least one of the right eye and the left eye.

6. The information processing apparatus according to claim 5, further comprising:
a dominant eye information acquiring unit for acquiring dominant eye information for specifying the dominant eye of the examinee,
wherein the risk assessing unit assesses the risk on the basis of the degree of coincidence of the dominant eye of the examinee specified by the dominant eye information.

7. The information processing apparatus according to any one of claims 1 to 6,
wherein the simulated moving image is a moving image that simulates a visual field of a person moving on the course while driving a vehicle.

8. An information processing system comprising:
an information processing apparatus according to any one of claims 1 to 7; and
the image display device.

9. The information processing system according to claim 8,
wherein the simulated moving image is composed of a right eye image and a left eye image, and
wherein the image display device is a head-mounted display including:
a right eye display executing unit for causing the right eye of the examinee to view the right eye image; and
a left eye display executing unit, provided independently of the right eye display executing unit, for causing the left eye of the examinee to view the left eye image.

10. An information processing method for assessing a risk of moving of an examinee, comprising the steps of:
acquiring head information for specifying movement of the head of the examinee;
causing an image display device to display a simulated moving image that is a moving image simulating a view of a person moving on a predetermined course, includes a scene including a target object, and changes according to the movement of the head of the examinee specified by the head information;
acquiring viewpoint information for specifying the position of the viewpoint of the examinee on the simulated moving image while the simulated moving image is displayed; and
assessing the risk on the basis of the degree of coincidence between the position of the viewpoint of the examinee specified by the viewpoint information and the position of the target object at the timing when the scene including the target object in the simulated moving image is displayed and outputting assessment information indicating the result of the assessment of the risk.

11. A computer program for assessing a risk of moving of an examinee, which causes a computer to perform:
a process of acquiring head information for specifying movement of the head of the examinee;
a process of causing an image display device to display a simulated moving image that is a moving image simulating a view of a person moving on a predetermined course, includes a scene including a target object, and changes according to the movement of the head of the examinee specified by the head information;
a process of acquiring viewpoint information for specifying the position of the viewpoint of the examinee on the simulated moving image while the simulated moving image is displayed; and
a process of assessing the risk on the basis of the degree of coincidence between the position of the viewpoint of the examinee specified by the viewpoint information and the position of the target object at the timing when the scene including the target object in the simulated moving image is displayed and outputting assessment information indicating the result of the assessment of the risk.
